(19) United States
(12) Patent Application Publication
(10) Pub. No.: US 2014/0224742 A1
(43) Pub. Date: Aug. 14, 2014

(54) PROCESS AND DEVICE FOR SIMULATED MOVING BED ADSORPTION AND SEPARATION WITH A REDUCED NUMBER OF CONTROL VALVES

(71) Applicants: Dehua Wang, Beijing (CN); Zhuo Yu, Beijing (CN); Huiguo Wang, Beijing (CN); Jianfeng Ma, Beijing (CN); Wei Wu, Beijing (CN)

(72) Inventors: Dehua Wang, Beijing (CN); Zhuo Yu, Beijing (CN); Huiguo Wang, Beijing (CN); Jianfeng Ma, Beijing (CN); Wei Wu, Beijing (CN)

(73) Assignees: CHINA PETROLEUM & CHEMICAL CORPORATION, Beijing (CN); RESEARCH INSTITUTE OF PETROLEUM PROCESSING, SINOPEC, Beijing (CN)

(21) Appl. No.: 14/131,955

(22) PCT Filed: Jul. 16, 2012
(86) PCT No.: PCT/CN2012/000955

§ 371 Date: Apr. 28, 2014

(30) Foreign Application Priority Data
Jul. 28, 2011 (CN) 201110213274.5

(51) Int. Cl. B01D 15/18

(52) U.S. Cl. CPC B01D 15/1835
USPC 210/670; 210/264

(57) ABSTRACT

The present invention relates to a process for simulated moving bed (SMB) adsorption and separation with a reduced number of control valves, comprising separating the feedstocks comprising isomers by SMB adsorption, said SMB comprising m beds of adsorbent, each of which is equipped with grids, each of the grids being equipped with the materials charging and discharging pipeline of the bed, the materials charged into and discharged from SMB at least comprising adsorption feedstocks, desorbent, extract, raffinate and the flushing liquids charged from different beds, wherein the to extract is enriched with the target product; there are at least two streams of said flushing liquid selected from any one of the adsorption feedstock, desorbent, extract and raffinate; there are altogether n streams of materials charged to and discharged from the SMB, wherein there are s types of materials having the same composition and flow direction; p sets of on-off valves are used to control n streams of materials is charged to and discharged from the beds of adsorbent, wherein at least one group of materials composed of two streams of materials having the composition and flow direction is controlled by one set of valves, with s≦p<n; in the operating process of the SMB, the total amount of the on-off valves used to control the charging and discharging of the materials is p×m. Said method can remarkably reduce the number of the on-off valves used in the operating process of SMB.

### TECHNICAL FIELD

The present invention relates to a process and device for separation of hydrocarbons by adsorption, specifically for separation and purification of hydrocarbons isomers by simulated moving bed (SMB) adsorption.

### BACKGROUND OF THE INVENTION

The adsorption separation is very effective for the separation of isomers having an extremely small boiling point difference, or of different components having different structural features, e.g. for the separation of p-xylene from other C₈ aromatic isomers, and of n-alkanes from hydrocarbons having other structures.

The separation process by SMB adsorption achieves countercurrent contact of the liquid and solid phases and increases the separation efficiency. U.S. Pat. No. 2,985,589, U.S. Pat. No. 3,201,491, U.S. Pat. No. 3,626,020, U.S. Pat. No. 3,686,342, U.S. Pat. No. 3,997,620 and U.S. Pat. No. 4,326,092 describe the separation device and process by SMB adsorption, and use thereof for separation of p-xylene and m-xylene. Douglas M. Ruthven summarizes in Chemical Engineering Science (1989, v44(5):1011-1038) the principle, development, test and model study and industrial process of the separation process by continuous countercurrent adsorption.

Typical SMB adsorption separation process comprises at least two streams of charged materials, i.e. feedstock (F) and desorbent (D), and at least two streams of the discharged materials, i.e. extract (E) and raffinate (R), wherein the extract is enriched with the target product. The positions at which each stream of the materials is charged to or discharged from the adsorption column are moved periodically, and the charging and discharging materials along with the flow direction of the materials in the adsorption column are in a sequence of the desorbent (D), extract (E), feedstock (F) and raffinmate (R). The circulation of materials in the adsorption column makes up an closed-loop. The device for controlling the charging and discharging of the materials to and from the adsorption column may be a rotary valve, or a series of on-off valves.

During the adsorption separation, many streams of materials share the delivery pipelines to be charged into or discharged from the adsorption column. The pipeline getting into and getting out of a certain bed position of the adsorption column will pass the raffinmate (R), feedstock (F), extract (E) and desorbent (D) in turn. The previous residual materials in the pipeline will pollute the extract.

U.S. Pat. No. 3,201,491 discloses a process for increasing the purity of the product obtained by continuous separation and adsorption. As for the circumstance that the residual feedstock pollutes the extract, it discloses “charging a flush stream comprising a fluid separable from said feed stock into the fluid inlet next upstream relative to the feed stream inlet in an amount not substantially exceeding the volume of fluid in the line of flow between the feed inlet into the fluid distribution center and the feed inlet to the contact zone receiving said feed stream”. The flushing liquid is a desorbent-rich stream removed from a fixed mass of sorbent downstream from the desorbent inlet, or a sorbate-rich stream withdrawn from the farthermost downstream mass of sorbent comprising the desorption zone.

U.S. Pat. No. 5,750,820 discloses a multiple grade flush adsorption separation process, which is a process for separating the target product from a multicomponent feedstream, comprising introducing said feedstream through at least one fluid communication conduit into said apparatus; flushing said apparatus having at least one fluid communication conduit with a sufficient quantity of at least one initial flushing medium drawn from a first source and comprising said at least one desired component in an initial concentration, such that feedstream residue is flushed from said apparatus by said at least one initial medium; flushing said at least one fluid communication conduit with a sufficient quantity of a final flushing medium drawn from a second source and comprising said at least one desired component in a final concentration, such that said final concentration is greater than said initial concentration and such that initial medium residue from said conduit is flushed into said apparatus by said final medium; and withdrawing said product from said apparatus, wherein said first source is different from said second source and at least one of said first source and said second source is separated from said adsorption separation apparatus.

U.S. Pat. No. 5,972,224 discloses a process and device for improving the purity of a product in a simulated fluid bed, said device comprising a number of beds (A₁ to Aₙ) equipped with a solid or adsorbent that are contained in at least one adsorption column, a fluid distributor plate (Pᵢ) between each bed, whereby each distributor plate is divided into a number of sectors (P₁₀, P₁₁, P₁₂), whereby each distributor plate sector (Pᵢ) includes at least one distribution chamber that is pierced with openings and a fluid circulation space in the vicinity of said openings of the chamber, and whereby said chamber is connected to a transfer line that extends between the chamber and a point that is located outside of the column; during a period T of the cycle, an injection and a draw-off of each feedstock into and from a distribution chamber that belongs to different plates are carried out, with the process being characterized in that, at an appropriate flow rate, a fluid volume is permanently circulated in a bypass line that connects different chambers of the distributor plates; the flushing liquid has a composition close to the circulating fluid. The object thereof lies in avoiding the disturbance to the separation process caused by greater composition difference between the flushing materials introduced from outside and the materials in the adsorption column. However, such solution will also cause a problem, i.e. a stream of the materials not passing through the adsorption chamber, which is equivalent to a stream of channeling in the bed of adsorbent, and is adverse to the adsorption separation.

CN200710139991.1 provides a solution of reducing the number of the valves: simulated moving bed (SMB) separation device comprises column, adsorbent bed Aᵢ separated by plate Pᵢ, the single distribution and extraction network of fluid, especially the feed F, the desorbent D, the raffinate R and the extract E, and several two-way valves used in distribution of said fluid; said column is divided into several segments Sk having 2 or 3 superimposed beds; each segment Sk comprises external bypass pipelines LK which are connected to each bed of Sk through the linking pipe containing the valves Vi corresponding to each and every bed; each pipeline LK comprises a control device limiting the internal flow thereof and is connected with each fluid network F, D, R and E through a single pipeline; said single pipeline comprises single controllable two-way isolation valves, which are used for providing corresponding fluid F, D, R or E to the considered segment Sk or taking corresponding fluid F, D, R or E from the considered segment Sk. Said solution can remarkably reduce the number of the valves but increase the complexity of control. Moreover, the valve fault of the fluid network leading to a certain segment will affect each and every bed of said segment, and the reliability of the system will thereby greatly decrease.

### SUMMARY OF THE INVENTION

The object of the present invention is to provide a process and device for simulated moving bed (SMB) adsorption and separation with a reduced number of control valves. Said process can greatly reduce the number of the control valves and remarkably reduce the installation investment under the condition of ensuring the purity, yield and treatment capacity of the target product of the adsorption separation.

The process for simulated moving bed (SMB) adsorption and separation with a reduced number of control valves provided by the present invention comprises separating the feedstocks comprising isomers by SMB adsorption, said SMB comprising m beds of adsorbent, each of which is equipped with grids, each of the grids being equipped with the materials charging and discharging pipeline of the bed, the materials charged into and discharged from SMB at least comprising adsorption feedstocks, desorbent, extract, raffinate and flushing liquids charged from different beds, wherein the extract is enriched with the target product; there are at least two streams of said flushing liquid selected from any one of the adsorption feedstock, desorbent, extract and raffinate; there are altogether n streams of materials charged to and discharged from the SMB, wherein there are s types of materials having the same composition and flow direction; p sets of on-off valves are used to control n streams of materials charged to and discharged from the beds of adsorbent, wherein at least one group of two streams of materials having the same composition and flow direction is controlled by one set of valves, and s≦p<n; in the operating process of the SMB, the total amount of the on-off valves used to control the charging and discharging of the materials is p×m.

The process of the present invention adsorbs and separates the isomers with a simulated moving bed, uses any one of the most basic charging and discharging materials of the SMB—the adsorption feedstock, the desorbent, the extract and the raffinate as the flushing liquid, and uses an on-off valve to control the charging and discharging materials of the SMB. There are altogether n streams of materials charged to and discharged from the SMB, wherein at least one group of materials having the same composition and flow direction is controlled by one set of valves, which thereby effectively reduces the number of the on-off valves of the SMB, reduces the number of the pipelines and optimizes the operation steps.

### DESCRIPTION OF THE DRAWINGS

FIG. 1 shows the diagrammatic representation of the setting of the valves controlling the charging and discharging materials to and from the adsorption column in one step time in Comparative Example 1.

FIG. 2 shows the diagrammatic representation of the setting of the valves controlling the charging and discharging materials to and from the adsorption column in one step time in Example 1 of the present invention.

FIG. 3 shows the diagrammatic representation of the setting of the valves controlling the charging and discharging materials to and from the adsorption column in one step time in Example 2 of the present invention.

FIG. 4 shows the diagrammatic representation of the setting of the valves controlling the charging and discharging materials to and from the adsorption column in one step time in Example 3 of the present invention.

FIG. 5 shows the diagrammatic representation of the setting of the valves controlling the charging and discharging materials to and from the adsorption column in one step time in Comparative Example 2.

FIG. 6 shows the diagrammatic representation of the setting of the valves controlling the charging and discharging materials to and from the adsorption column in one step time in Example 4 of the present invention.

### DETAILED DESCRIPTION OF THE INVENTION

The process of the present invention controls two streams of materials, in the SMB, having the same composition and flow direction and charged to and discharged from the bed of adsorbent with the same one valve, and controls the amount of the material charged to the bed of adsorbent by controlling the turn-on time of the control valve, and thereby reduces the number of the on-off valves used in the SMB for controlling the charging and discharging of the materials.

The SMB used for separation by adsorption according to the present process comprises one or more adsorption columns, each of which is separated with grids to many beds of adsorbent, wherein said grids have the following functions: re-distributing the materials from the previous bed to the next bed, homogeneously mixing the materials introduced from outside with the materials from the previous bed, and withdrawing a part of the materials from the previous bed from the adsorption column. The grids permit the passage of the liquid and intercept the adsorbent particles from escaping from the bed of adsorbent, wherein the upper and lower surfaces thereof are generally the woven wire cloth, metal sintering screen or Johnson Screen.

The materials introduced from outside to a certain bed, and the materials withdrawn from previous bed and out of the adsorption column are charged to and discharged from the bed of adsorbent via a pipeline connected with grids of the bed.

The materials charged to and discharged from the adsorption column in the process of the present invention at least comprise the feedstock (F), desorbent (D), extract (E) raffinate (R) and at least a flushing liquid. The feedstocks are the mixtures comprising at least two or more components which include the target product purified by adsorption separation. Each component in the feedstocks has different selectivities regarding the adsorbents, and the adsorbents have higher adsorption selectivity regarding the target products. The desorbent is be greatly different from the feedstocks in the boiling point, and may be separated from the components in the feedstocks by rectification. The extract is enriched with the target product and meanwhile comprises a part of the desorbent. The raffinate comprises a small amount of the target product. The less the content thereof is, the higher the adsorption separation efficiency is. The main components of the raffinate are the desorbent and other components in the feedstocks except the target product. The desorbent is separated respectively from the extract and raffinate by the rectification column and recycled.

The charging and discharging materials along with the flow direction of the materials in the adsorption column are in a sequence of the desorbent (D), extract (E), feedstock (F) and raffinate (R). The beds of adsorbent between the desorbent inlet and the extract outlet are the desorption zone. The beds of adsorbent between the extract outlet and the feedstock inlet are the purification zone. The beds of adsrobent between the feedstock inlet and the raffinate outlet are the adsorption zone. The beds of adsorbent between the raffinate outlet and the desorbent inlet are the isolation zone. The simulated moving bed has from 6 to 30 beds, preferably from 12 to 24 beds. Two adsorption columns are generally used, totaling 24 beds, wherein 4-6 beds are the desorption zone; 8-10 beds are the purification zone; 6-8 beds are the adsorption zone; 2-3 beds are the isolation zone. In the present invention, the upstream and downstream of the charging and discharging positions of some certain materials at which the flushing liquid is charged to are relative to the bed at the charging and discharging positions of said materials in the adsorption column. It is the downstream along with the flow direction of the materials in the adsorption column, and the opposite is the upstream. For example, the flushing liquid is charged to one bed at the downstream of the extract withdrawal position, i.e. the flushing liquid is charged to the next bed of the extract withdrawal position along with the flow direction of the materials of the bed.

In order to minimize the volume of the residual materials need to be flushed, the process of the present invention does not adopt a rotary valve to control the flow of the materials, but connects each charging and discharging materials to the pipelines linked to the grids and controls said materials with respective on-off valves, which enables the on-off valves to get close to the adsorption column to the greatest extent and thereby reduces the volume of the pipelines.

To remove the impact of the residual materials in the delivery pipelines of the materials on the adsorption separation process, the adsorption bed is flushed with flushing liquids. Therefore, a SMB has n streams of charging and discharging materials, m beds of adsorbent, s groups of materials having the same composition and flow direction, each group composed of two streams of materials. At a certain moment, among the on-off valves connecting each stream of charging and discharging material with different beds, at most n−1 on-off valves and at least s on-off valves are in an open state, and the others ones are in a closed state. At a certain interval, i.e. one step time, the position of each charging and discharging material move one bed downward. One step time is preferably 45˜200 seconds.

In the process of the present invention, n is the number of the streams of the materials charged to and discharged from the SMB, n is preferably an integer of 6˜8, p is the number of the materials charged to and discharged from the adsorption bed after combining the two streams of materials having the same composition and flow direction, p is preferably an integer of 5˜7, m is an integer of 12˜30. After combination, n streams of charging and discharging materials are charged to each bed of adsorbent through p pipelines controlled with p on-off valves.

One solution of the present invention is to set two streams of flushing liquids which are the extract, are respectively charged to 1˜2 beds at the upstream of the feedstock charging position and the 2˜4 beds at the downstream of the extract withdrawing position, and respectively used to remove the impact of the feedstock (F) remains in the pipelines on the extract (E). Said two streams of flushing liquids are charged to the bed of adsorbent through the pipelines controlled with the same set of on-off valves. The flushing liquid charged to 2˜4 beds at the downstream of the extract withdrawing positioned is the second flushing liquid. The flushing liquid charged to 1˜2 beds at the upstream of the feedstock charging position is the third flushing liquid. Said two streams of flushing liquids are conducive to obtaining highly pure target product. It is referred to as the purification zone flushing to set two streams of flushing liquids at the two ends of the purification zone, which are respectively close to the positions of the feedstock and the extract, and to flush inward the adsorption bed with said liquids.

In the process of SMB separation process, the extract (E) that remains in the pipelines is flushed by the desorbent (D) into the boundary of the desorption zone and the isolation zone, which leads to the decrease of the yield. The desorbent may be flushed inward the bed in the desorption zone at a position close to the extract, or flushed outward by withdrawing the materials from the bed at the position close to the charging position of the desorbent, which is referred to as the desorption zone flushing. The process of the present invention preferably sets the first flushing liquid in the desorption zone, the component of which is desorbent and the charging position is 1-2 beds of adsorbent at the upstream of the extract withdrawing point. The first flushing liquid and the desorbent are charged to the bed of adsorbent through the pipelines controlled by the same set of on-off valves. Two streams of flushing materials are set in the purification zone, which use the extract as the flushing liquid and are respectively charged to 1˜2 beds at the upstream of the feedstock charging position and 2˜4 beds at the downstream of the extract withdrawing position. Said two streams of flushing liquids are charged to the bed of adsorbent through the pipelines controlled with the same set of on-off valves. The flushing liquid charged to 2˜4 beds at the downstream of the extract withdrawing position is the second flushing liquid. The flushing liquid charged to 1˜2 beds at the upstream of the feedstock charging position is the third flushing liquid.

In the process of the SMB separation process, the raffinate (R) that remains in the pipelines is flushed by the feedstock (F) into the adsorption zone to reduce the adsorption volume of the target product. The feedstock may be flushed inward the bed in the adsorption zone at a position close to the raffinate or flushed outward the beds of adsorbent at the position close to the feedstock, which is referred to as the adsorption zone flushing. To minimize the factors affecting the separation effect, the present invention preferably sets the first flushing liquid in the desorption zone, the component of which is desorbent, and the charging position is 1˜2 beds of adsorbent at the upstream of the extract withdrawing point; sets the fourth flushing liquid in the adsorption zone, the component of which is the feedstock, and the charging position is 1˜2 beds at the upstream of the raffinate withdrawing point. The first stream of the flushing liquid and the desorbent are charged to the bed of adsorbent through the pipelines controlled by the same set of valves. The feedstock and the fourth stream of the flushing liquid are charged to the bed of adsorbent through the pipelines controlled by the same set of valves. Two streams of flushing liquids are set in the purification zone. The extract is used as the flushing liquid. Said two streams of the flushing liquids are respectively charged to 1˜2 beds at the upstream of the feedstock charging position and 2˜4 beds at the downstream of the extract withdrawing position. Said two streams of flushing liquids are charged to the bed of adsorbent through the pipelines controlled by the same set of valves. The flushing liquid charged to 2˜4 beds at the downstream of the raffinate withdrawing position is the second flushing liquid, and the flushing liquid charged to 1˜2 beds at the upstream of the feedstock charging position is the third flushing liquid.

The volume of the second flushing liquid as disclosed in the present process is 0.5˜1.5 times of the total volume of the pipelines from the control valves to the bed of adsorbent. The volume of the third flushing liquid is 1.0˜2.5 times of the total volume of the pipelines from the control valves to the bed of adsorbent.

The volume of the first flushing liquid is 0.7˜1.5 times of the total volume of the pipelines from the control valves to the bed of adsorbent.

The volume of the fourth flushing liquid is 0.6˜1.0 times of the total volume of the pipelines from the control valves to the bed of adsorbent.

According to the process of the present invention, the incorporated materials having the same composition are charged to different beds of adsorbent via the same set of valves through which the same main pipeline leads to different beds. For instance, the desorbent and the first flushing liquid are delivered with one main pipeline. At a certain moment, the beds that need the desorbent and the first flushing liquid are two different beds. The desorbent is then charged in two streams to the different beds through the desorbent control valves of the beds. The same set of valves of the present invention refers to a group of valves having the same tag in each bed, for example, the valves tagged with D/C1 in each bed are the same set of on-off valves.

In the present invention, although the two streams of materials have the same composition and flow direction, the volumes thereof needed in one step time are different. There are two methods able to realize the different charging volumes. One method is to set the time when the on-off valves leading to the corresponding bed in an open state according to the volume of the materials needed, within one step time. The on-off valves corresponding to the bed needing larger volume of materials are open for a longer time. That is to say, the volume of the flushing liquid shall be controlled by the turn-on time of the valve controlling said flushing liquid charged to and discharged from said bed, within one step time. Another method is to respectively set flow control valves in the routes through which the materials are charged to different beds; keep the on-off valves of the beds corresponding to the two streams sharing the same set of on-off valves in a open state in one step time; control the volume by the flow control valves set in the corresponding beds according to the volume of the materials needed by each bed.

The application device of process of the present invention comprises a simulated moving bed comprising m beds of adsorbent, each of which is equipped with grids, each of the grids being equipped with the materials charging and discharging pipeline of the bed, wherein the material charging and discharging pipelines are connected with p inlet and outlet pipelines, said p inlet and outlet pipelines are connected in parallel, one on-off valve is set in each pipeline, and in the adsorption separation process, there are n streams of materials charged to and discharged from the SMB, wherein there are s types of materials having the same composition and flow direction, and s≦p<n, wherein n is an integer of 6˜8, p is an integer of 5˜7 and m is an integer of 12˜30.

To control the volume of the material charged to the bed of adsorbent, a flow control valve is set in the pipeline by which two streams of materials go in each bed, where the flow of the materials is controlled by the opening degree thereof.

The adsorption separation process to which the process of the present invention is applicable is a liquid-phase adsorption separation process. The adsorption separation temperature is preferably 20˜300° C., and the operating pressure shall ensure that the system is a complete liquid phase.

The isomers absorbed and separated in the present invention are preferably xylenes and ethylbenzene. The target product of the adsorption separation is preferably para-xylene or meta-xylene. The desorbent used in the adsorption separation is preferably p-diethylbenzene or toluene.

When p-xylene is separated from the mixture of C8 aromatic isomers, the purity of the product is required to be at least 99.5 mass %, and preferably above 99.7 mass %. The desorbent is preferably p-diethylbenzene (PDEB), and the adsorbent is preferably faujasite exchanged with barium or/and potassium, and is preferably X-type zeolite.

Two adsorption columns are generally used, totalling 24 beds, wherein 4-6 beds are the desorption zone; 8-10 beds are the purification zone; 6-8 beds are the adsorption zone; 2-3 beds are the isolation zone. The operating temperature is 120˜190° C., and the operating pressure is 0.8˜1.2 MPa.

When m-xylene (MX) is separated from the mixture of C8 aromatic isomers, the purity of the product is required to be at least 99.5 mass %, and preferably above 99.7 mass %. The desorbent is preferably toluene, and the adsorbent is preferably faujasite exchanged with alkali metal ions, and is preferably Y-type zeolite. Two adsorption columns are generally used, totaling 24 beds, wherein 4-6 beds are the desorption zone; 8-10 beds are the purification zone; 6-8 beds are the adsorption zone; 2-3 beds are the isolation zone. The operating temperature is 100˜180° C., and the operating pressure is 0.8˜1.2 MPa.

The present invention is further disclosed in, but not limited to, the following examples.

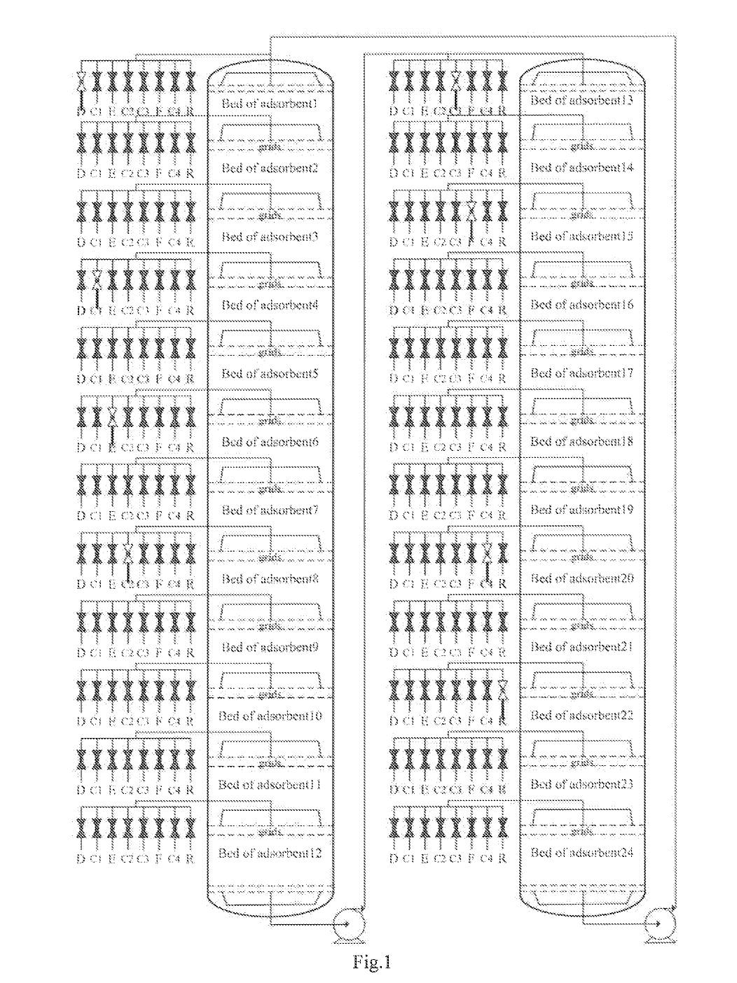

FIG. 1-6 show the valves open and closed in each bed within one step time, wherein the white coreless valves represent the open valves; the black solid valves represent the closed valves; the English letters below the valves represent the materials controlled by each valve; D is the desorbent; E is the extract; F is the feedstock; and R is the raffinate.

### Comparative Example 1

SMB has 24 adsorption beds, wherein the desorption zone has 5 beds; the purification zone has 9 beds; the adsorption zone has 7 beds; the isolation zone has 3 beds. The operating temperature was 177. The operating pressure was 0.88 MPa. The feedstock is mixed xylenes containing ethylbenzene, comprising 18.4 wt % PX, 44.5 wt % MX, 20.2 wt % OX (o-xylene), 12.1 wt % ethylbenzene, and the other components including alkane or naphthene having 8-9 carbon atoms, a small amount of toluene and aromatic hydrocarbon having 9 carbons. The target product of adsorption separation is PX. The adsorbent is RAX-2000A-type adsorbent produced by SIPOPEC Catalyst Company, with the main component being X-type molecular sieves exchanged with barium ions. A first flushing (C₁) is set, which uses the desorbent as the flushing liquid and is charged to the second bed at the upstream of the extract withdrawing point. A second flushing (C₂), wherein the extract is used as the flushing liquid, is charged to the second bed at the downstream of the extract withdrawing point. A third flushing (C₃), wherein the extract is used as the flushing liquid, is charged to the second bed at the upstream of the feedstock charging position. A fourth flushing (C₄), wherein the feedstock is used as the flushing liquid, is charged to the second bed at the upstream of the extract withdrawing point. One step time is 80 seconds.

There are in total 8 streams of materials charged to and discharged from the whole stimulated moving adsorption bed. An individual on-off value is set up for each stream of the materials charged to/discharged from each bed. Each bed of adsorbent has 8 pipelines, wherein 8 on-off valves are set. 8 pipelines are connected to materials charging and discharging pipelines in the grids of said bed. 24×8=192 on-off valves are needed to control the charging and discharging of the materials in each adsorption bed. The layout of the valves of each bed of adsorbent of the SMB within one step time is shown in FIG. 1;

The volume of the pipelines need to be flushed is 0.04 m³. The ratio of the volume of the first flushing liquid to the volume of the pipelines need to be flushed is 1.2. The ratio of the volume of the second flushing liquid to the volume of the pipelines need to be flushed is 1.0. The ratio of the volume of the third flushing liquid to the volume of the pipelines need to be flushed is also 1.2. The ratio of the volume of the fourth flushing liquid to the volume of the pipelines need to be flushed is 0.9. Thus, in one step time, the volume of the first flushing liquid is 0.048 m³; the volume of the second flushing liquid is 0.04 m³; the volume of the third flushing liquid is 0.048 m³; the volume of the fourth flushing liquid is 0.036 m³. The flows of all materials are as the follows: feedstock (F), 28.28 m³/h; desorbent (D), 35.76 m³/h; extract (E) which is withdrawn from the adsorption column, 19.69 m³/h. However, since a part of the extract goes back as the materials of the second flushing and the third flushing, the flow of the extract which is actually left for the subsequent separation steps is 15.73 m³/h, the flow of the first flushing (C1) is 2.16 m³/h, the flow of the second flushing (C2) is 1.8 m³/h, the flow of the third flushing (C3) is 2.16 m³/h, and the flow of the fourth flushing (C4) is 1.62 m³/h. The flow of the raffinate is controlled by the system pressure, so as to ensure the overall balance of the charging and the discharging of the materials.

The device operation results show that the purity of the product is 99.72%, and the yield is 97.3%.

### Example 1

The process of the present invention is used to separate p-xylene. The feedstock, desorbent, adsorbent, the operating temperature and the operating pressure are identical to those in Comparative Example 1. The SMB, the number of the beds of adsorbent in each zone and the charging positions of the four flushing liquids are all identical to those in Comparative Example 1.

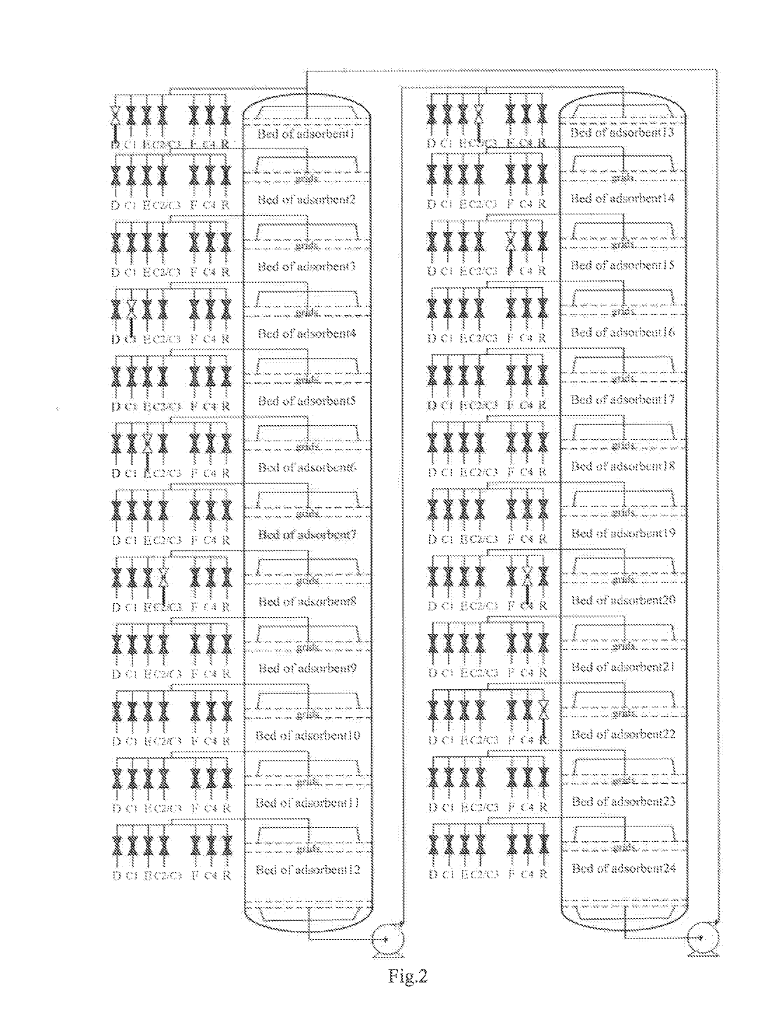

According to the setting form of the valves as shown in FIG. 2, the materials of the second flushing and the third flushing are delivered through the same main pipeline, the flow of which is controlled with a main flow control valve. Said materials are charged to each bed through the same set of on-off valves, namely, being charged to the bed need to be flushed through a C₂/C₃ common valve of each bed. 24×7=168 on-off valves are needed to control the charging and discharging of each stream of materials of SMB.

The volume of the pipelines need to be flushed at every turn is 0.04 m³. The ratio of the volume of the first flushing material to the volume of the pipelines need to be flushed is 1.2. The ratio of the volume of the second flushing material to the volume of the pipelines need to be flushed is 0.9. The ratio of the volume of the third flushing material to the volume of the pipelines need to be flushed is also 1.2. The ratio of the volume of the fourth flushing material to the volume of the pipelines need to be flushed is 0.9. Thus, in one step time, the volume of the first flushing liquid needed is 0.048 m³; the volume of the second flushing liquid is 0.036 m³; the volume of the third flushing liquid is 0.048 m³; the volume of the fourth flushing liquid is 0.036 m³. The step time is 75 seconds. Compared with the Comparative Example 1, the step time is shorter, the circulation velocity of the adsorbent is accelerated and the amount of the adsorption feedstock is increased in the same proportion. The flows of all materials are as follows: feedstock (F), 30.18 m³/h; desorbent (D), 38.16 m³/h; extract (E) which is withdrawn from the adsorption column, 20.82 m³/h. However, since a part of the extract goes back as the materials of the second flushing and the third flushing, the flow of the extract which is actually left for the subsequent separation steps is 16.79 m³/h, the flow of the first flushing (C1) is 2.3 m³/h, the total flow of the second flushing (C2) and the third flushing (C3) is 4.03 m³/h, and the flow of the fourth flushing (C4) is 1.73 m³/h.

The following contents describe how to control the materials with the same set of valves so that they can get into different positions with a required volume.

FIG. 2 marks the turning-on conditions of the on-off valves in the pipelines of each adsorption bed in one step time. At 0 second, the valve D that controls the desorbent, which is connected with the grids above the adsorption bed 1 is open, the first flushing valve C1 connected with the grids above the bed 4 is open, the extract valve E connected with the grids above the bed 6 is open, the common valve C2/C3 connected with the grids above the bed 8 is open to allow the second flushing liquid to flow in, the feedstock control valve F connected with the grids above the bed 15 is open, the fourth flushing valve C4 connected with the grids above the bed 20 is open, the raffinate valve R connected with the grids above the bed 22 is open, and the other valves are in a closed state. At the 32^{nd} second, the common valve C2/C3 connected with the grids above the bed 13 is turned on to allow the third flushing liquid to flow in, the common valve C2/C3 connected with the grids above the bed 8 is turned off. At the 75^{th} second, the positions of feedstock, the desorbent, the extract, the raffinate, C1 and C4 are all shifted to the next bed. The specific operation steps of the valves are as follows: the desorbent valve D connected with the grids above the bed 2 is turned on, and the desorbent valve D connected with the grids above the bed 1 is turned off; the valve C1 connected with the grids above the bed 5 is turned on, and the valve C1 connected with the grids above the bed 4 is turned off; the extract valve E connected with the grids above the bed 7 is turned on, and the extract valve E connected with the grids above the bed 6 is turned off; the feedstock valve F connected with the grids above the bed 16 is turned on, and the feedstock valve F connected with the grids above the bed 15 is turned off; the valve C4 connected with the grids above the bed 21 is turned on, and the valve C4 connected with the grids above the bed 20 is turned off; the raffinate valve R connected with the grids above the bed 23 is turned on, and the raffinmate valve R connected with the grids above the bed 22 is turned off. The situation of the common valve C2/C3 is as follows: the common valve C2/C3 connected with the grids above the bed 9 is turned on, and the common valve C2/C3 connected with the grids above the bed 13 is turned off; at the 75+32^{nd} second, the common valve C2/C3 connected with the grids above the bed 14 is turned on, and the common valve C2/C3 connected with the grids above the bed 9 is turned off. The material on-off valves in the pipelines of each bed are operated in the same way in each step time.

Compared with the situation as shown in Comparative Example 1, a total of 24 less on-off valves are used, and the operating results show that the purity of the product is 99.71% and the yield is 97.0%, which are not obviously different from the results in Comparative Example 1.

### Example 2

The process of the present invention is used to separate p-xylene. The feedstock, desorbent, adsorbent, the operating temperature and the operating pressure are identical to those in Comparative Example 1. The SMB, the number of the beds in each zone and the charging positions of the four flushing liquids are all identical to those in Comparative Example 1.

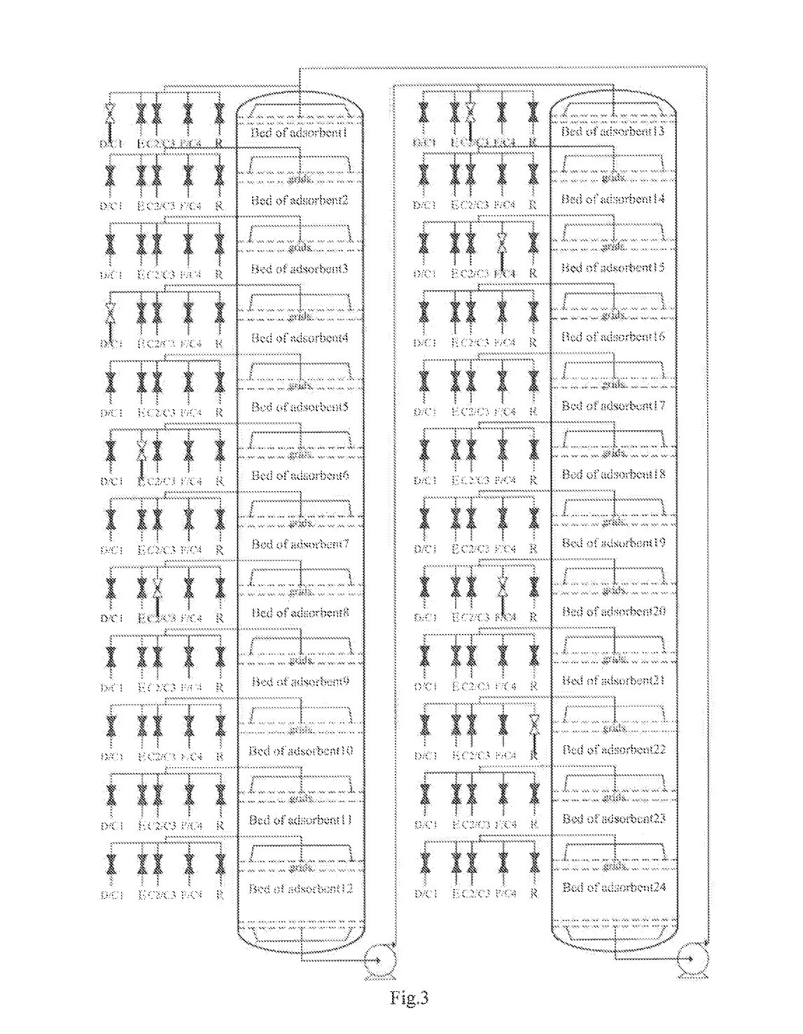

According to the setting form of the valves as shown in FIG. 3, the desorbent and the materials of the first flushing are delivered through the same main pipeline, the flow of which is controlled with a main flow control valve; the desorbent and the first flushing liquid are both charged to the required adsorption beds through the same set of on-off valves D/C1; the second flushing liquid and the third flushing liquid are delivered through the same main pipeline, the flow of which is controlled with a main flow control valve; the second flushing liquid and the third flushing liquid are both charged to the required beds through the same set of on-off valves C2/C3; the feedstock and the fourth flushing liquid are delivered through the same main pipeline, the flow of which is controlled with a main flow control valve; the feedstock and the fourth flushing liquid are both charged to the required beds through the same set of on-offvalves F/C4; on-offvalves are set respectively for the other extract and raffinate charged to each bed; a total of 24×5=120 on-off valves are needed to control the charging and discharging of 8 streams of materials of SMB.

The volume of the pipelines need to be flushed at every turn is 0.04 m³. One step time is 80 seconds. The flushing ratio of the first flushing is 1.0. The flushing ratio of the second flushing is 1.0. The flushing ratio of the third flushing is 1.5. The flushing ratio of the fourth flushing is 0.9. Thus, in one step time, the volume of the first flushing liquid is 0.04 m³; the volume of the second flushing liquid is 0.04 m³; the volume of the third flushing liquid is 0.06 m³; the volume of the fourth flushing liquid is 0.036 m³.

The flow needed by the desorbent is 35.77 m³/h. The volume of the liquid needed in the first flushing amounts to a flow of 1.80 m³/h in one step time. Then, the total flow in the common pipeline of the desorbent and the first flushing is controlled according to the sum of the two 37.57 m³/h. The flow in the common pipeline of the second flushing and the third flushing shall enable the volume of the liquid that passes in one step time to reach the sum of the two 0.10 m³, wherein the flow is 4.50 m³/h. The flow needed by the feedstock is 28.28 m³/h. The volume of the liquid needed in the fourth flushing amounts to a flow of 1.62 m³/h in one step time. The total flow in the common pipeline of the feedstock and the fourth flushing is controlled according to the sum of the two 29.90 m³/h. The flow of the extract (E) which is withdrawn from the adsorption column is 19.33 m³/h. However, since a part of the extract goes back as the materials of the second flushing and the third flushing, the flow of the extract which is actually left for the subsequent separation step is 14.83 m³/h.

The flowing contents describe how to control the materials with the same set of valves so that they can get into different positions with a required volume.

In one step time, the common valve D/C1 through which the desorbent is charged to the corresponding bed always remains open; the valve E through which the extract leaves the corresponding bed always remains open; the common valve C2/C3 through which the third flushing reaches the corresponding bed always remains open; the common valve F/C4 through which the feedstock reaches the corresponding bed always remains open; the valve R through which the extract leaves the corresponding bed always remains open. The common valve D/C1 through which the first flushing reaches the corresponding bed is open for 7.7 seconds in one step time and remains closed in the rest of the time. The common valve C2/C3 through which the second flushing reaches the corresponding bed is open for 64 seconds in one step time and remains closed in the rest of the time. The common valve F/C4 through which the fourth flushing reaches the corresponding bed is open for 8.7 seconds in one step time and remains closed in the rest of the time.

FIG. 3 marks the on-off conditions of the valves in each bed in one step time. At 0 second, the valve D/C1 connected with the grids above the bed 1 is open to allow the desorbent to flow in, the extract valve E connected with the grids above the bed 6 is open, the common valve C2/C3 connected with the grids above the bed 13 is open, the valve F/C4 connected with the grids above the bed 15 is open to the feedstock to flow in, the valve R connected with the grids above the bed 22 is open, raffinate outflows, and the other valves are in a closed state. At a certain moment, e.g. at the 8^{th} second, the common valve C2/C3 connected with the grids above the bed 8 is open for 64 seconds to flush said bed for the second time and is turned off at the 8+64=72^{nd} second. At a certain moment, e.g. at the 20^{th} second, the common valve D/C1 connected with the grids above the bed 4 is open for 7.7 seconds to flush said bed for the first time and is turned off at the 20+7.7=27.7^{th} second. At a certain moment, e.g. at the 20^{th} second, the common valve F/C4 connected with the grids above the bed 20 is open for 8.7 seconds to flush said bed for the fourth time and is turned off at the 20+8.7=28.7^{th} second. At the 80^{th} second, the positions of feedstock, the desorbent, the extract, the raffinate and the flushing liquid C3 are all shifted to the next bed. The specific operation steps of the valves are as follows: the common valve D/C1 connected with the grids above the bed 2 is turned on, and the common valve D/C1 connected with the grids above the bed 1 is turned off; the extract valve E connected with the grids above the bed 7 is turned on, and the extract valve E connected with the grids above the bed 6 is turned off; the common valve C2/C3 connected with the grids above the bed 14 is turned on, and the common valve C2/C3 connected with the grids above the bed 13 is turned off; the common valve F/C4 connected with the grids above the bed 16 is turned on, and the common valve F/C4 connected with the grids above the bed 15 is turned off; the raffinate valve R connected with the grids above the bed 23 is turned on, and the raffinate valve R connected with the grids above the bed 22 is turned off; the first, second and fourth flushing liquids are also correspondingly moved downward a bed, and the turn-off time of the corresponding valves are identical to the charging time of the liquids needed by each bed before said liquids are moved downward. The rest can be done in the same manner.

As for a certain bed, the method for controlling the inlet and outlet of each stream of charging and discharging materials is as follows: at the 0 moment, the common valve D/C1 leading to said bed is turned on, the desorbent begins to get into said bed through the pipeline connected to the grids above said bed, at this time, said bed is in the desorption zone; after one step time 80 seconds, the valve D/C1 is turned off, the desorbent stops getting into said bed and gets into the next bed. No material is charged to or discharged from the original bed which lies in the isolation zone. At the 3×80^{th} second, the valve R connected to the grids above said bed is turned on, the raffinate begins to leave the adsorption column through the pipeline connected to the grids above said bed. At the 4×80^{th} second, the raffinate valve connected to the grids above said bed is turned off; the raffinate begins to leave said bed through the pipeline connected to the grids below said bed; said bed enters the adsorption zone. At the (5×80+20)^{nd} second, the common valve F/C4 leading to said bed is turned on to conduct the C4 flushing. At the (5×80+28.7)^{th} second, said valve is turned off. At the 10×80^{th} second, the common valve F/C4 leading to said bed is turned on; the feedstock begins to enter said bed through the pipeline connected to the grids above said bed; at this time, said bed still lies in the adsorption zone. At the 11×80^{th} second, the common valve F/C4 leading to said bed is turned off; the common valve C2/C3 leading to said bed is turned on to conduct the C3 flushing; at this time, said bed enters the purification zone; at the 12×80^{th} second, the common valve C2/C3 leading to said bed is turned off. At the (5×80+28.7)^{th} second, the common valve C2/C3 leading to said bed is turned on to conduct the C2 flushing. At the (17×80+72)^{nd} second, the common valve C2/C3 leading to said bed is turned off. At the 19×80^{th} second, the extract valve E connected to the grids above said bed is turned on; the extract begins to leave the adsorption column through the pipeline connected to the grids above said bed. At the 21×80^{th} second, the extract valve E connected to the pipeline in the grids above said bed is turned off; the extract begins to leave the adsorption column through the pipeline connected to the grids below said bed, at this time, said bed enters the desorption zone. At the (21×80+20)^{nd} second, the common valve D/C1 leading to said bed is turned on to conduct the C1 flushing. At the (21×80+27.7)^{th} second, said valve is turned off. At the 24×80^{th} second, the common valve D/C1 leading to said bed is turned on; the desorbent once again enters said bed; thus a complete circulation is completed.

Compared with the situation as shown in Comparative Example 1, a total of 72 less on-off valves are used, and the operating results show that the purity of the product is 99.74% and the yield is 96.9%, which are not obviously different from the results in Comparative Example 1.

### Example 3

The process of the present invention is used to adsorb and separate p-xylene. The feedstock, desorbent, adsorbent, the operating temperature and the operating pressure are identical to those in Comparative Example 1. The SMB and the number of the bed layers in each zone are both identical to those in Comparative Example 1. The first flushing (C1) is set up, which is the desorbent that is charged to the second bed at the upstream of the extract withdrawing point. The second flushing (C2) is the extract and charged to the second bed at the downstream of the extract withdrawing point. The third flushing (C3) is the extract and charged to the second bed at the upstream of the feedstock charging point. Like Comparative Example 1, the fourth flushing is not set.

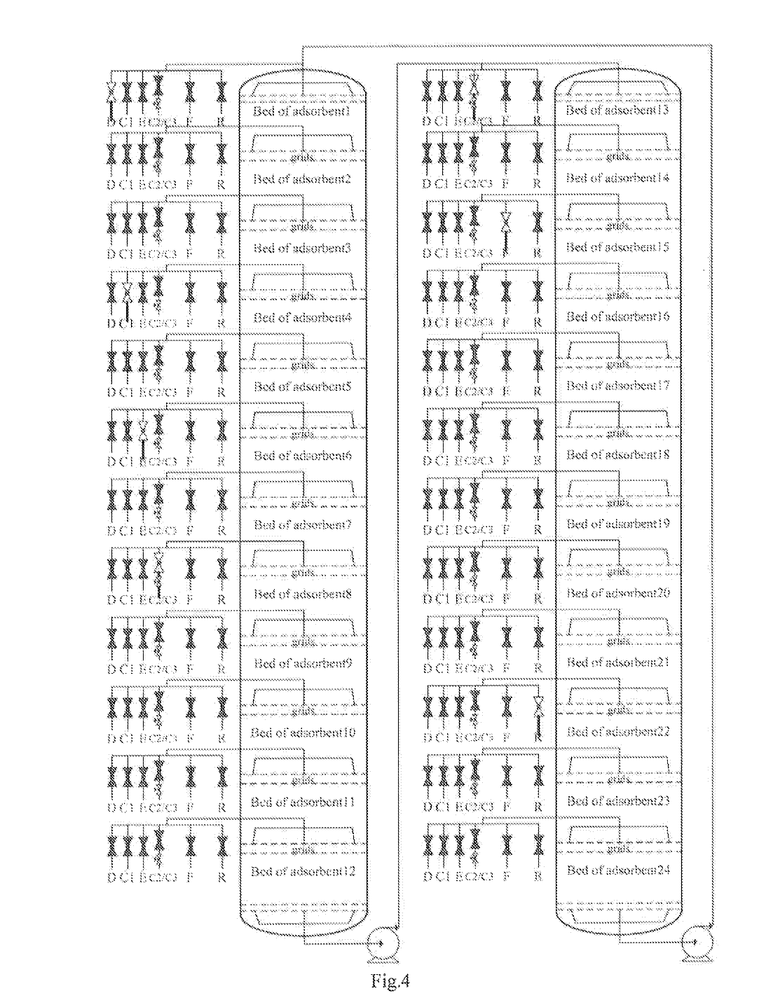

The valves are set according to FIG. 4, the materials of the second flushing and the third flushing are delivered through the same main pipeline, the flow of which is controlled with a main flow control valve. Said materials are charged to each bed through the same set of on-off valve C₂/C₃. A flow control valve is set in a C2/C3 common pipeline leading to each bed. On-off valves are respectively set for other each stream of materials charged to each bed. A total of 24×6=144 on-off valves are needed to control the charging and discharging of 7 stream of materials.

The volume of the pipelines need to be flushed is 0.04 m³. One step time is 75 seconds. Compared with the Comparative Example 1, the step time is shorter, the circulation velocity of the adsorbent is accelerated and the amount of the adsorption material is increased in the same proportion. The first flushing ratio is 1.0. The second flushing ratio is 0.9. The third flushing ratio is 1.2. Thus, in one step time, the volume of the first flushing liquid is 0.04 m³; the volume of the second flushing liquid is 0.036 m³; the volume of the third flushing liquid is 0.048 m³. The flows of all materials are as follows: feedstock (F), 31.9 m³/h; desorbent (D), 38.16 m³/h; extract (E) which is withdrawn from the adsorption column, 20.43 m³/h. However, since a part of the extract goes back as the materials of the second flushing and the third flushing, the flow of the extract which is actually left for the subsequent separation step is 16.40 m³/h, the flow of the first flushing (C1) is 1.92 m³/h, the total flow of the second flushing (C2) and the third flushing (C3) is 4.03 m³/h.

The following contents disclose how to control two streams of feedstocks with the same one set of valves and set different opening degrees of the flow control valves in the branch pipelines leading to each bed so as to enables the required volume get into different positions.

FIG. 4 shows the on-off conditions of the valves of each adsorption bed in one step time. At 0 second, the desorbent valve D connected with the grids above the bed 1 is open; the valve C1 connected with the grids above the bed 4 is open; the extract valve E connected with the grids above the bed 6 is open; the common valve C2/C3 connected with the grids above the bed 8 is open; the common valve C2/C3 connected with the grids above the bed 13 is open; the feedstock valve F connected with the grids above the bed 15 is open; the raffinmate valve R connected with the grids above the bed 22 is open; and the other valves are in a closed state, wherein the opening degree of the valve adjusting the flow of the second flushing charged to the bed 8 is different from the opening degree of the valve adjusting the flow of the third flushing charged to the bed 13; the opening degree of the valve adjusting the flow of the second flushing charged to the bed 8 is relatively small, so that the flow of the second flushing is the target flow 1.73 m³/h; the opening degree of the valve adjusting the flow of the third flushing charged to the bed 13 is relatively large, so that the flow of the third flushing is the target flow 2.30 m³/h. At the 75^{th} second, the positions of the feedstock, the desorbent, the extract, the raffinate and C1, C2 and C3 flushings are all shifted to the next bed. The specific operation steps of the valves are as follows: the desorbent valve D connected with the grids above the bed 2 is turned on, and the desorbent valve D connected with the grids above the bed 1 is turned off; the valve C1 connected with the grids above the bed 5 is turned on, and the valve C1 connected with the grids above the bed 4 is turned off; the extract valve E connected with the grids above the bed 7 is turned on, and the extract valve E connected with the grids above the bed 6 is turned off; the common valve C2/C3 connected with the grids above the bed 9 is turned on, and the common valve C2/C3 connected with the grids above the bed 8 is turned off; the common valve C2/C3 connected with the grids above the bed 14 is turned on, and the common valve C2/C3 connected with the grids above the bed 13 is turned off; the feedstock valve F connected with the grids above the bed 16 is turned on, and the feedstock valve F connected with the grids above the bed 15 is turned off; the raffinate valve R connected with the grids above the bed 23 is turned on, and the raffinate valve R connected with the grids above the bed 22 is turned off; before the shift, the opening degree of the flow control valve in common valve C2/C3 leading to bed 9 and 14 is adjusted beforehand to be identical to that of the flow control valve in common valve C2/C3 of the corresponding beds before the shift, and the opening degree of the flow control valve leading to bed 14 is larger than that of the flow control valve leading to bed 9.

Since the fourth flushing C4 is not set, the yield is reduced. The operating results show that the purity of the product is 99.71%, and the yield is 94.9%.

### Comparative Example 2

The process for adsorbing and separating p-xylene according to the prior art: the SMB, the number of the beds in each zone; the adsorption feedstock, the adsorbent, the desorbent, the operating temperature and the operating pressure are identical to those in Comparative Example 1. The first flushing (C1) is set, which rushes outward from the adsorption column at the first bed at the downstream of the desorbent charging point. The second flushing (C2), which uses the desorbent as the flushing liquid, is charged to the first bed at the downstream of the extract withdrawing point. The third flushing (C3), which uses the materials withdrawn in the first flushing as the flushing liquid, is charged to the second bed at the upstream of the feedstock charging point. The fourth flushing (C4), which uses the feedstock as the flushing liquid, is charged to the second bed at the upstream of the raffinate withdrawing point. One step time is 80 seconds. The volume of the pipelines need to be flushed is 0.04 m³. The ratio of the volume of the first flushing material to the volume of the pipelines need to be flushed is 1.2. The ratio of the volume of the second flushing material to the volume of the pipelines need to be flushed is 1.0. The volume of the third flushing material, which is identical to the volume of the first flushing material, is 1.2 times of the volume of the pipelines need to be flushed. The ratio of the volume of the fourth flushing material to the volume of the pipelines need to be flushed is 0.8. The flows of all materials are as follows: the feedstock (F), 28.46 m³/h; the desorbent (D), 35.76 m³/h; the extract (E), 19.69 m³/h; the first flushing (C1), 2.16 m³/h; the second flushing (C2), 1.8 m³/h; the third flushing (C3), 2.16 m³/h; the fourth flushing (C4), 1.44 m³/h.

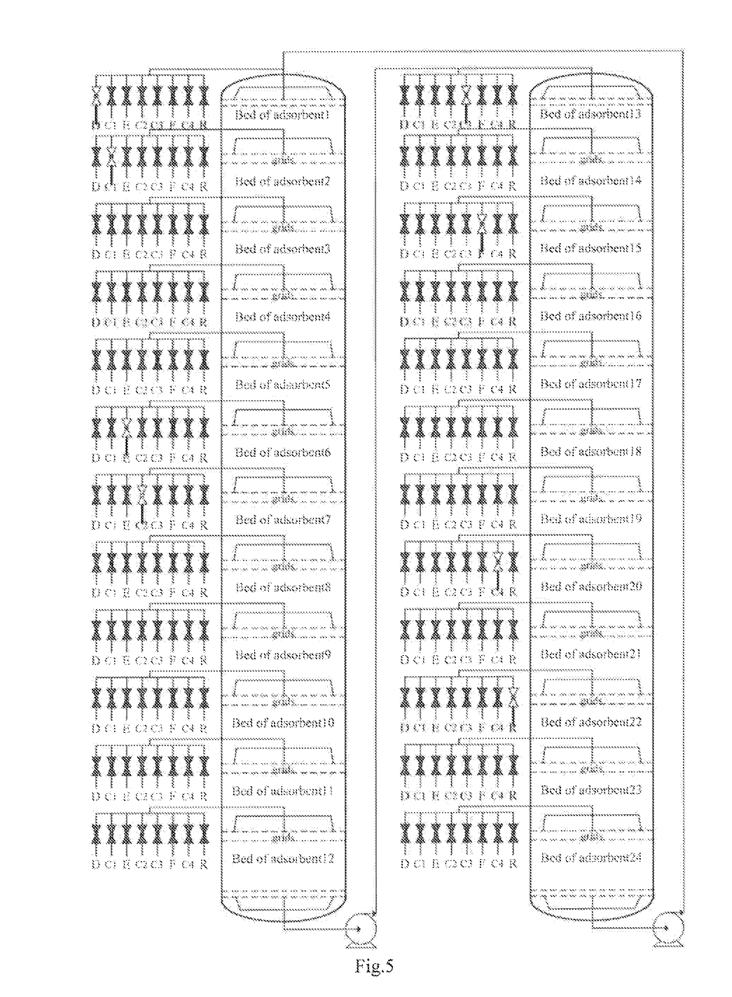

There are in total 8 streams of materials charged to and discharged from the whole stimulated moving adsorption bed. An individual on-off value is set up for each stream of charged and discharged materials into/from each bed. Each bed has 8 pipelines, wherein 8 on-off valves are set. 8 pipelines are connected to the materials charging and discharging pipelines in the grids of said bed. A total of 24×8=192 on-off valves are needed to control the charging and discharging of the materials in each bed. The layout of the valves of each bed of the SMB within one step time is shown in FIG. 5. The purity of the product is 99.71%, and the yield is 92%.

### Example 4

The process of the present invention is used to adsorb and separate p-xylene. The SMB, the number of the beds in each zone, the adsorption feedstock, the adsorbent, the desorbent, the operating temperature and pressure, the step time and the positions and the volume of the four flushings are identical to those in Comparative Example 2.

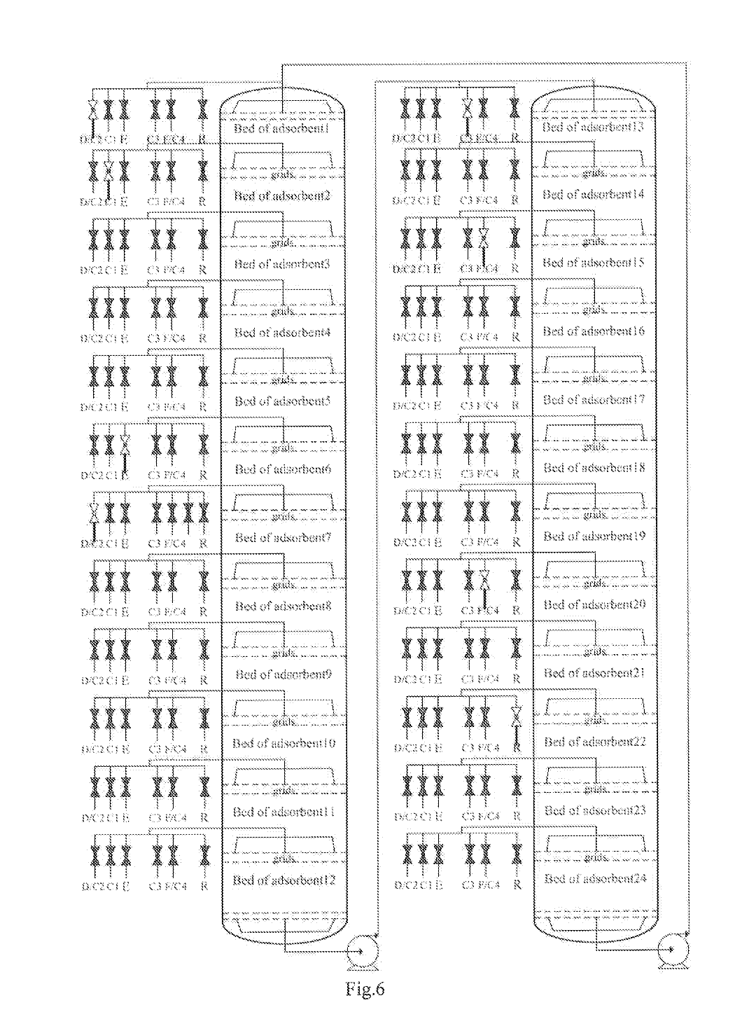

According to the setting form of the valves as shown in FIG. 6, the desorbent and the materials of the second flushing are delivered through the same main pipeline, the flow of which is controlled with a main flow control valve; said desorbent and the second flushing liquid are both charged to the required beds through the same set of on-off valve D/C2; the feedstock and the fourth flushing liquid are delivered through the same main pipeline, the flow of which is controlled with a main flow control valve; said feedstock and the fourth flushing liquid are charged to the required beds through the same set of on-off valve F/C4; On-off valves are respectively set for other raffinate, extract, the first flushing and the third flushing charged to each bed. A total of 24×6=144 on-off valves are needed to control the charging and discharging of 8 streams of materials of the SMB.

The flow of each material is as follows: the total volume of the feedstock and the fourth flushing (F/C4) is 29.9 m³/h; the total volume of the desorbent and the second flushing (D/C2) is 37.56 m³/h; the volume of the extract (E) is 19.69 m³/h; the volume of the first flushing (C1) is 2.16 m³/h; the volume of the third flushing (C3) is 2.16 m³/h. The following contents describe how to control the materials with the same one set of valves so as to enables the materials get into different positions with required volumes.

FIG. 6 shows the on-off conditions of the valves in the pipelines of each bed in one step time. In one step time, the common valve D/C2 through which the desorbent is charged to the corresponding bed always remains open; the valve C1 through which the first flushing reaches the corresponding bed always remains open; the valve E through which the extract leaves the corresponding bed always remains open; the valve C3 through which the third flushing reaches the corresponding bed always remains open; the common valve F/C4 through which the feedstock is charged to the corresponding bed always remains open; the valve R through which the extract leaves the corresponding bed always remains open; the common valve D/C2 through which the second flushing reaches the corresponding bed remains open for 7.67 seconds within one step time and remains closed in the rest of the time; the common valve F/C4 through which the fourth flushing reaches the corresponding bed is open for 7.71 seconds in one step time and remains closed in the rest of the time.

At 0 second, the valve D/C2 connected with the grids above the bed of adsorbent 1 is open to allow the desorbent to flow in, the first flushing C1 valve connected with the grids above the bed 2 is open, the extract valve E connected with the grids above the bed 6 is open, the third flushing C3 valve connected with the grids above the bed 13 is open, the valve F/C4 connected with the grids above the bed 15 is open to let the feedstock flow in, the valve R connected with the grids above the bed 22 is open, raffinate outflows, and the other valves are turned off. At a certain moment, e.g. the 8^{th} second, the common valve D/C2 connected with the grids above the bed 7 is open for 7.67 seconds to flush said bed for the second time and is turned off at the 8+7.67=15.67^{th} second. At a certain moment, e.g. the 20^{th} second, the common valve F/C4 connected with the grids above the bed 20 is open for 7.71 seconds to flush said bed with the fourth flushing and is turned off at the 20+7.71=27.71^{st} second. At the 80^{th} second, the positions of the feedstock, the desorbent, the extract, the raffinate, the first flushing C1 and the third flushing C3 are all shifted to the next bed. The specific operation steps of the valves are as follows: the common valve D/C2 connected with the grids above the bed 2 is turned on, and the common valve D/C2 connected with the grids above the bed 1 is turned off; the first flushing C1 valve connected with the grids above the bed 3 is turned on, and the first flushing C1 valve connected with the grids above the bed 2 is turned off; the extract valve E connected with the grids above the bed 7 is turned on, and the extract valve E connected with the grids above the bed 6 is turned off; the valve C3 connected with the grids above the bed 14 is turned on, and the valve C3 connected with the grids above the bed 13 is turned off; the common valve F/C4 connected with the grids above the bed 16 is turned on, and the common valve F/C4 connected with the grids above the bed 15 is turned off; the raffinate valve R connected with the grids above the bed 23 is turned on, and the raffinate valve R connected with the grids above the bed 22 is turned off; the positions of the second and fourth flushings are moved downward one bed; the common valve D/C2 connected with the grids above the bed 8 is turned on at the 88^{th} second and turned off at the 95.67^{th} second; the common valve F/C4 connected with the grids above the bed 21 is turned on at the 100^{th} second and turned off at the 107.71^{st} second; in the same manner, all the rest valves are moved downward one bed after every step time.

As for a certain bed, the method for controlling the on-off valves controlling the inlet and outlet of each stream of charging and discharging material is as follows: at the 0 moment, the common valve D/C2 leading to said bed is turned on, the desorbent begins to get into said bed through the pipeline connected to the grids above said bed, at this time, said bed is in the desorption zone; after one step time 80 seconds, the valve D/C2 is turned off, the desorbent stops getting into said bed and gets into the next bed; no material is charged to or discharged from the original bed, which lies in the isolation zone; at the 3×80^{th} second, the valve R connected to the pipeline in the grids above said bed is turned on, the raffinate begins to leave the adsorption column through the pipeline connected to the grids above said bed; at the 4×80^{th} second, the raffinate valve connected to the pipeline in the grids above said bed is turned off; the raffinate begins to leave said bed through the pipeline connected to the grids below said bed; said bed enters the adsorption zone; at the 5×80+20^{th} second, the common valve F/C4 connected to the grids above said bed is turned on to conduct the C4 flushing; at the (5×80+27.71)^{st} second, said valve is turned off; at the 10×80^{th} second, the common valve F/C4 connected to the grids above said bed is turned on, the feedstock begins to enter said bed through the pipeline connected to the grids above said bed, at this time, said bed still lies in the adsorption zone; at the 11×80^{th} second, the common valve F/C4 connected to the grids above said bed is turned off, at this time, said bed enters the purification zone; at the 12×80^{th} second, the valve C3 connected to the grids above said bed is turned on to conduct the C3 flushing; at the 13×80^{th} second, the valve C3 connected to the grids above said bed is turned off; at the (18×80+8)^{h} second, the common valve D/C2 connected to the grids above said bed is turned on to conduct the C2 flushing; at the (18×80+15.67)^{th} second, the common valve C2/C3 leading to said bed is turned off; at the 19×80^{th} second, the extract valve E connected to the grids above said bed is turned on, the extract begins to leave the adsorption column through the pipeline connected to the grids above said bed; at the 21×80^{th} second, the extract valve E connected to the pipeline in the grids above said bed is turned off, the extract begins to leave said bed through the pipeline connected to the grids below said bed, at this time, said bed enters the desorption zone; at the 23×80^{th} second, the valve C1 connected to the grids above said bed is turned on to conduct the C1 flushing and turned off at the 24×80, second; at the 24×80^{th} second, the common valve D/C2 leading to said bed is turned on, the desorbent once again enters said bed, thus a complete circulation is completed.

Compared with the situation as shown in Comparative Example 2, a total of 48 less on-off valves are used. The operating results show that the purity of the product is 99.71% and yield is 91.8%, which are not obviously different from the results in Comparative Example 2.

The situations of the comparative examples and the examples are concluded in the following table.

| | No. | | | | | |
|---|---|---|---|---|---|---|
| | Comparative | | | | Comparative | |
| | Example 1 | Example 1 | Example 2 | Example 3 | Example 2 | Example 4 |
| number of | 8 | 8 | 8 | 7 | 8 | 8 |
| streams of | | | | | | |
| materials | | | | | | |
| number of | 24 * 8 = 192 | 24 * 7 = 168 | 24 * 5 = 120 | 24 * 6 = 144 | 24 * 8 = 192 | 24 * 6 = 144 |
| valves | | | | | | |
| Common valves | null | C2/C3 | D/C1 | C2/C3 | null | D/C2 |
| | | | C2/C3 | | | F/C4 |
| | | | F/C4 | | | |
| manner of using | | within one | the stream | in one step | | the stream |
| common valves | | step time, | with the | time, the | | with the |
| | | the valve | larger | valve leading | | larger flow |
| | | leading to | flow (D, | to the C2 | | (D, F) in |
| | | the C2 | C3, F) in | charging | | the two |
| | | charging | the two | position and | | streams |
| | | position is | streams | the valve | | that share |
| | | open in | that share | leading to the | | the valve |
| | | the first | the valves | C3 charging | | is |
| | | part 0-32 | is | position are | | completely |
| | | seconds, | completely | both open, | | open |
| | | and closed | open | however, the | | within one |
| | | in the | within one | opening | | step time, |
| | | second | step time, | degrees of the | | and the |
| | | part 32-75 | and the | adjusting | | stream |
| | | seconds | stream | valves | | with |
| | | | with | leading to the | | smaller |
| | | | smaller | bed layer are | | flow (C2, |
| | | | flow is | different, the | | C4) is |
| | | | open in a | opening | | open in a |
| | | | part of the | degree of C3 | | part of the |
| | | | time | which needs a | | time |
| | | | | larger flow is | | |
| | | | | larger | | |
| the step time, | 80 | 75 | 80 | 75 | 80 | 80 |
| seconds | | | | | | |
| general flow of | 29.9 | 31.91 | 29.9 | 31.9 | 29.9 | 29.9 |
| the feedstock | | | | | | |
| (F + C4) | | | | | | |
| C1 | 1.2 | 1.2 | 1.0 | 1.0 | 1.2 | 1.2 |
| flushing ratio | | | | | | |
| C2 flushing | 1.0 | 0.9 | 1.0 | 0.9 | 1.0 | 1.0 |
| ratio | | | | | | |
| C3 flushing | 1.2 | 1.2 | 1.5 | 1.2 | 1.2 | 1.2 |
| ratio | | | | | | |
| C4 flushing | 0.9 | 0.9 | 0.9 | — | 0.8 | 0.8 |
| ratio | | | | | | |
| purity, % | 99.72 | 99.71 | 99.74 | 99.71 | 99.71 | 99.71 |
| yield, % | 97.3 | 97 | 96.8 | 94.9 | 92 | 91.8 |

1. A process for simulated moving bed (SMB) adsorption and separation with a reduced number of control valves, comprising separating the feedstocks comprising isomers by SMB adsorption, said SMB comprising m beds of adsorbent, each of which is equipped with grids, each of the grids being equipped with the materials charging and discharging pipeline of the bed, the materials charged into and discharged from SMB at least comprising adsorption feedstocks, desorbent, extract, raffinate and flushing liquids charged from different beds, wherein the extract is enriched with the target product; there are at least two streams of said flushing liquid selected from any one of the adsorption feedstock, desorbent, extract and raffinate; there are altogether n streams of materials charged to and discharged from the SMB, wherein there are s types of materials having the same composition and flow direction; p sets of on-off valves are used to control n streams of materials charged to and discharged from the beds of adsorbent, wherein at least one group of two streams of materials having the same composition and flow direction is controlled by one set of valves, and s≦p<n; in the operating process of the SMB, the total amount of the on-off valves used to control the charging and discharging of the materials is p×m.

2. A process according to claim 1, characterized in that n is an integer of 6˜8, p is an integer of 5˜7, m is an integer of 12˜30, and n streams of charging and discharging materials are passed to each bed of adsorbent through p pipelines controlled with p valves.

3. A process according to claim 1, characterized in that the extracts is used as the flushing liquids which are respectively charged to 1˜2 beds at the upstream of the feedstock charging position and 2˜4 beds at the downstream of the extract withdrawing position, two streams of flushing liquids get into the bed of adsorbent through the pipelines controlled by the same set of valves, the flushing liquid charged to 2˜4 beds at the downstream of the extract withdrawing position is the second flushing, and the flushing liquid charged to 1˜2 beds at the upstream of the feedstock charging position is the third flushing liquid.

4. A process according to claim 3, characterized in setting the first flushing liquid, the component of which is desorbent, wherein the charging position is 1˜2 beds at the upstream of the extract withdrawing position, and the first flushing liquid and the desorbent are charged to the beds of adsorbent through pipelines controlled with the same set of valves.

5. A process according to claim 3, characterized in setting the first flushing liquid, the component of which is desorbent, wherein the charging position is 1˜2 beds at the upstream of the withdrawing position of the extract; setting the fourth flushing liquid, the component of which is the feedstocks, wherein the charging position is 1˜2 beds at the upstream of the raffinate withdrawing position, the first flushing liquid and the desorbent are charged to the bed of adsorbent through pipelines controlled with the same set of valves, and the feedstocks and the fourth stream of the flushing liquid are charged to the bed of adsorbent through pipelines controlled with the same set of valves.

6. A process according to any one of claims 3˜5, characterized in that the amount of the second flushing liquid is 0.5˜1.5 times of the total volume of the pipeline from the controlling valve to the bed of adsorbent, through which the flushing liquid passes, and the amount of the third flushing liquid is 1.0˜2.5 times of the total volume of the pipeline from the controlling valve to the bed of adsorbent, through which the third flushing liquid passes.

7. A process according to claim 4 or 5, characterized in that the amount of the first flushing liquid is 0.7˜1.5 times of the total volume of the pipeline from the controlling valve to the bed of adsorbent through which the first flushing liquid passes.

8. A process according to claim 5, characterized in that the amount of the fourth flushing liquid is 0.6˜1.0 times of the total volume of the pipeline from the controlling valve to the bed of adsorbent through which the fourth flushing liquid passes.

9. A process according to claim 1, characterized in that the incorporated materials having the same composition are charged to different beds of adsorbent via the same set of valves through which the same main pipeline leads to different beds of adsorbent.

10. A process according to claim 1, characterized in that the amount of the flushing liquid is controlled by the turn-on time of the valves controlling the flushing liquid charged to flush the beds or the flow of said flushing liquid during one step time.

11. A process according to claim 1, characterized in that said adsorption and separation process is a liquid phase adsorption and separation process.

12. A process according to claim 1, characterized in that the isomers adsorbed and separated are xylenes and ethylbenzene, and the target product of adsorption and separation is para-xylene or meta-xylene.

13. A process according to claim 1, characterized in that the desorbent used in the adsorption and separation is para-diethylbenzene or toluene.

14. The application device of the process according to claim 1, comprising a simulated moving bed comprising m beds of adsorbent, each of which is equipped with grids, each of the grids being equipped with the materials charging and discharging pipeline of the bed, wherein the materials charging and discharging pipelines are connected with p inlet and outlet pipelines, said p inlet and outlet pipelines are connected in parallel, one valve is set in each pipeline, and in the adsorption separation process, there are n streams of materials charged to and discharged from the SMB, wherein there are s types of materials having the same composition and flow direction, and s≦p<n.

15. The device according to claim 14, characterized in that n is an integer of 6˜8, p is an integer of 5˜7, m is an integer of 12˜30.

16. The device according to claim 14, characterized in that a flow control valve is set in the pipelines in each bed of adsorbent, through which two streams of materials pass.
